(19) United States
(12) Patent Application Publication
(10) Pub. No.: US 2002/0082705 A1
(43) Pub. Date: Jun. 27, 2002

(54) Basal finger joint implant

(76) Inventors: Hans-Werner Bouman, Heidelberg (DE); Hans-Georg Pfaff, Ostfildern (DE); Gontran Sennwald, Gallen (CH); Markus Schwarz, Ebenhausen (DE)

Correspondence Address: FULBRIGHT & JAWORSKI, LLP, 666 FIFTH AVE, NEW YORK, NY, 10103-3198, US

(21) Appl. No.: 09/960,624

(22) Filed: Sep. 21, 2001

(30) Foreign Application Priority Data
Sep. 22, 2000 (DE) 100 47 033.5
Sep. 7, 2001 (DE) 101 43 865.6

(51) Int. Cl. A61F 2/42

(52) U.S. Cl. 623/21.11

(57) ABSTRACT

According to the state of the art, the replacement of a basal finger joint is effected by, for example, interposition of soft part tissue or a distance piece, which does not, however, constitute a replacement joint. In joint prostheses with a metal-metal sliding combination, but in particular with a metal/polyethylene sliding combination, wear of the material can lead to osteolyses at the bone ends. Owing to the tact that the metal or plastic joint prostheses known from the state of the art are as a rule multipart, loosening of the individual components can occur, which shortens the service life of the implant.

According to the invention, it is therefore proposed that the implant (1) is an uncoupled, two-part implant.

The invention relates to a basal finger joint implant.

According to the state of the art, the replacement of a basal finger joint is effected by, for example, interposition of soft part tissue or a distance piece made of a plastic, for example Silastik®. Most frequently, a silicone Spacer is used, which only serves as a distance piece and does not constitute a replacement joint. After its insertion, the joint is unstable and only very limitedly movable Premature wear of the material and as a result osteolyses at the bone ends can occur. In joint prostheses with a metal-metal sliding combination, but in particular with a metal/polyethylene sliding combination, wear of the material can lead to osteolyses at the bone ends. Owing to the fact that the metal or plastic joint prostheses known from the state of the art are as a rule multipart, loosening of the individual components can occur, which shortens the service life of the implant. As the basal finger joint implants, the finger joint endoprostheses, are as a rule coupled, this often leads to premature loosening of the joint.

The object of the present invention is to produce an artificial basal finger joint which makes possible anatomical movement in a wear-free sliding combination. Over and above this, biologically inert materials are to make possible long-term joint replacement.

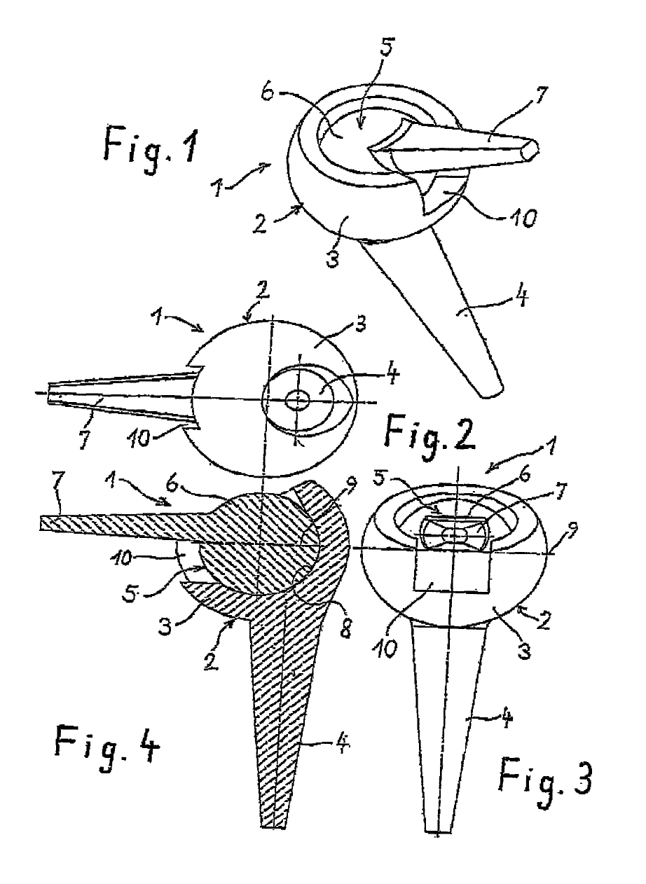

The object is achieved by an uncoupled, two part implant with congruent, spherical sliding surfaces. It is illustrated in FIGS. 1 to 4.

FIG. 1 shows a perspective view. The basal finger joint implant 1 consists of two monolithic components, the proximal component 2, consisting of the hollow-ball-shaped socket bearing 3 with the proximal shaft 4 , and the distal component 5, consisting of a ball 5 which is mounted in the socket bearing 3 and implanted in the finger bone by means of the distal shaft 7.

FIG. 2 shows the proximal View.

FIG. 3 shows a view of the implant in flexion position, and

FIG. 4 shows a section through the implant in the position according to FIG. 3 in a side view.

As can be seen from FIG. 4, the bearing surface 8 of the socket bearing 3 extends beyond the equatorial plane 9 and thus affords great luxation protection that is protection against dislocations of the phalanx. On full extension of the phalanges, abduction/adduction of up to +/−30 angular degrees is possible. Adduction is ensured by a cutout 10, which is suitable for movement, in the proximal component 2. As flexion increases, in other words as bending of the finger increases, the implant is guided in such a manner that both abduction and adduction are increasingly restricted.

The advantages of the implant according to the invention reside in the fact that it consists entirely of ceramic, preferably of aluminum oxide ceramic. The proximal and the distal implant components are both monolithic. The shape of the prosthesis makes possible good mobility with anatomical lateral guidance. The shape or the bearing and the material itself guarantee high wear resistance and thus long-term durability Implantaton is effected without cement. To this end, the proximal shaft 4 and the distal shaft 7 have a coating, for example hydroxyapatite, which promotes bone ingrowth, or osteointegration. The shafts can also have a structure which is porous and thus favors ingrowth of bone tissue.

On account of its shape, the prosthesis according to the invention is especially suitable for the replacement of basal finger joints which have been destroyed or are unstable, in particular in rheumatics

1. A basal finger joint implant, wherein the implant (1) is an uncoupled, two-part implant

2. The basal finger joint implant as claimed in claim 1, wherein the implant (1) consists of two monolithic components, the proximal component (2), consisting of the hollow-ball-shaped socket bearing (3) with the proximal shaft (4), and the distal component (5), consisting of a ball (6) which is mounted in the socket bearing (3) and is implanted in the finger bane by means of the distal shaft (7).

3. The basal finger joint implant as claimed in claim 1 or 2, wherein the implant (1) has congruent, spherical sliding surfaces, one of which is the hollow-ball-shaped socket bearing (3) and the other of which is the surface of the ball (6).

4. The basal finger joint implant as claimed in one of claims 1 to 3, wherein the bearing surface (8) of the socket bearing (3) extends beyond the equatorial plane (9) as protection against luxation.

5. The basal finger joint implant as claimed in one of claims 1 to 4, wherein adduction is ensured by a cutout (10), which is suitable for movement, in the proximal component (2), and, on fall extension of the phalanges, abduction/adduction of up to +/−30 angular degrees is possible.

6. The basal finger joint implant as claimed in one of claims 1 to 5, wherein, as flexion increases, in other words as bending of the finger increases, the guidance of the distal shaft (7) is designed in such a manner that both abduction and adduction are increasingly restricted.

7. The basal finger joint implant as claimed in one of claims 1 to 6, which consists entirely of ceramic.

8. The basal finger joint implant as claimed in claim 7, which consists entirely of aluminum oxide ceramic 9

9. The basal finger joint implant as claimed in one of claims 1 to 8, wherein the proximal shaft (4) and the distal shaft (7) have a coating which promotes bone ingrowth, or osteointegration.

10. The basal finger joint implant as claimed in claim 9, wherein the coating promoting bone ingrowth is hydroxyapatite.

11. The basal finger joint implant as claimed in one of claims 1 to 8, wherein the proximal shaft (4) and the distal shaft (7) have a porous structure which promotes bone ingrowth, or osteointegration.
